(19) United States
(12) Patent Application Publication
(10) Pub. No.: US 2012/0065733 A1
(43) Pub. Date: Mar. 15, 2012

(54) INTER-VERTEBRAL IMPLANT HAVING DRAIN CAVITIES THERETHROUGH

(76) Inventor: Brian Howard Wieder, Casper, WY (US)

(21) Appl. No.: 12/881,041

(22) Filed: Sep. 13, 2010

(51) Int. Cl. A61F 2/44

(52) U.S. Cl. 623/17.11

(57) ABSTRACT

An inter-vertebral implant having drainage cavities therethrough is disclosed. Drainage of surgical byproducts, spinal fluid and blood, and administration of medicine or cooled saline to the volume between the inter vertebral implant and the spinal cord are enabled during and after surgery.

### FIELD OF THE INVENTION

This invention relates generally to inter-vertebral implants and, more particularly, to inter-vertebral implants having drain cavities effective for draining fluids away from or injecting fluids into a spinal cord region.

### BACKGROUND OF THE INVENTION

Spinal inter-vertebral implants include devices such as prosthetics, spacers, cages, artificial discs, and structural supports. Such implants are mechanical or static. Inter-vertebral implants are made from a variety of materials, including, cobalt chromium, titanium, stainless steel, poly ethyl-ethyl-ketone, metallic alloys and non-metallic materials, including absorbable and non-absorbable, polymers and bone grafts. These implants are typically surgically inserted between two spinal vertebral bodies to replace a removed disc or vertebral body, and can be collectively referred to as spinal inter-vertebral implants.

Inter-vertebral implants include manufactured, biomechanically complex devices to simple forms, including a piece of bone or plastic polymer that functions as a spacer between vertebral bodies. Inter-vertebral implants may be used to reconstitute normal biomechanical saggital and coronal contours of the spine, decompress the spinal cord and nerve roots via distraction, and replace a poorly functioning inter-vertebral disc with a mechanically functional device that allows for motion preservation, as examples.

Spinal inter-vertebral implants are surgically introduced either anterior to the inter-vertebral space or posterior through the spinal canal, after the inter-body disc or vertebral components are first removed surgically. The discs or components are then replaced with a spinal inter-vertebral implant of choice.

Spinal epidural hemotoma, or fluid collection, is the accumulation of blood products or cerebrospinal fluid in the epidural space, which lies between an implanted device and the spinal cord and/or its coverings and is a known surgical complication, which may occur when inter-vertebral devices are implanted. Although rare, it may be catastrophic when it occurs, leading to a rapidly progressive neurological injury. If such injury is not emergently addressed, it may lead to spinal cord compression with resultant spinal cord injury, including permanent quadriplegia or paraplegia. For this reason, patients remain hospitalized overnight under observation.

Optimal inter-vertebral implant size is typically dictated by patients' specific anatomy, where the strongest portion of the vertebral body, the apophyseal ring, is placed in contact with the inter-vertebral implant. The apophyseal ring is disposed towards the periphery of the vertebral body.

Sizing of the inter-vertebral implant to overlap with the apophyseal ring carries known risks. If the surgery requires complete removal of the inter-body disc and the posterior longitudinal ligament, there is nothing between the inter-vertebral body spacer and the spinal cord to protect the spinal cord from being compressed. Post surgical byproducts, including any blood products, spinal fluid or post surgical ooze from the adjacent tissues may accumulate in the space between the inter-vertebral implant and the spinal cord. Smaller inter-vertebral implants may be selected to allow for efflux of post-surgical byproducts that accumulate in the space between the inter-vertebral implant and the spinal cord around the inter-vertebral implant. Although such implant size reduction reduces the risk of spinal cord injury, it also prevents the use of inter-vertebral implants, which match the sizes of the apophyseal rings.

The use of a smaller inter-vertebral implant is not without risk. Occasionally, surrounding tissues swell post-operatively, which may cause occlusion of the naturally occurring cavities for allowing efflux of accumulating post-surgical byproducts, which may also lead to a compressive hemotoma affecting the spinal cord. Additionally, once inter-vertebral implant is positioned, any bleeding that occurs during surgery is difficult to control without removing the inter-vertebral implant.

In cases of traumatic spinal cord injury, recent studies have identified a clinical benefit of cooling spinal cord tissue following a trauma. Mechanisms for doing this include total body hypothermia, and local application of cold saline onto the damaged spinal cord. Both methods for achieving spinal cord cooling are highly inefficient and complicated making clinical application of these methods less acceptable. Nonetheless, spinal cord cooling in the face of spinal cord injury is a known clinical benefit.

### SUMMARY OF THE INVENTION

Accordingly, it is an object of embodiments of the present invention to provide an inter-vertebral implant effective for reducing the accumulation of blood, cerebral-spinal fluid and other post-surgical byproducts in the spinal column area, resulting from disc removal and injections.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

To achieve the foregoing and other objects, and in accordance with the purposes of the present invention, as embodied and broadly described herein, the inter-vertebral implant hereof includes: a wall having a chosen height defining an upper surface and a lower surface, an inside surface of the wall enclosing a bone growth volume extending through the upper surface and lower surface, an outside surface of the wall forming an anterior portion, a posterior portion opposite thereto, a first lateral portion between the anterior portion and the posterior portion, and a second lateral portion between the anterior portion and the posterior portion opposite the first lateral portion, the first lateral portion having a cavity therethrough having an orifice in both the anterior portion and the posterior portion of the wall, and the second lateral portion having a cavity therethrough having an orifice in both the anterior portion and the posterior portion of the wall.

In another aspect of the invention as embodied and broadly described herein, the inter-vertebral implant includes: a wall having a chosen height defining an upper surface and a lower surface, an internal surface partially enclosing two opposing bone growth volumes extending through the upper surface and the lower surface, a portion of the wall separating the bone growth volumes, and an outside surface of the wall forming an anterior portion, a posterior portion opposite thereto, the portion of the wall separating the opposing bone growth volumes having at least one cavity therethrough having an orifice in both the anterior portion and the posterior portion of the wall.

In another embodiment of the present invention, and in accordance with its objects and purposes, a method for reducing and removing the accumulation of fluids from the site of the spinal cord and applying material to the spinal cord region is presented. The method includes the step of inserting an inter-vertebral implant between two vertebrae of the spinal column, the implant comprising a wall having a chosen height defining an upper surface and a lower surface, an inside surface at least partially enclosing one or more bone growth volumes extending through the upper surface and the lower surface, and an outside surface of the wall forming an anterior portion, a posterior portion opposite thereto, and a portion of the wall separating the anterior and posterior portions of the wall and having at least one cavity therethrough having a orifices in both the anterior portion and the posterior portion of the wall.

Benefits and advantages of the present invention include, but are not limited to, providing an apparatus and method for reducing the accumulation of blood, cerebral-spinal fluid and other post-surgical byproducts in the region of the spinal column area resulting from disc removal, and for enabling the administration of thrombotic agents, cooled saline, medicines, and other materials to the spinal column during and after surgical processes.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a front perspective view of one embodiment of the present invention.

FIG. 2 illustrates a rear perspective view of the embodiment of the present invention shown in FIG. 1 hereof.

FIG. 3 illustrates a front planar view of the embodiment of the present invention shown in FIGS. 1 and 2 hereof.

FIG. 4 illustrates a side planar view of the embodiment of the present invention shown in FIGS. 1 and 2 hereof, disposed between two vertebrae.

FIG. 5 illustrates a planar top view of one embodiment of the present invention below a vertebra.

FIG. 6 illustrates a top perspective view of one embodiment of the present invention shown in FIGS. 1 and 2 hereof.

FIG. 7 illustrates a front perspective view of the embodiment of the present invention shown in FIGS. 1 and 2 hereof showing ridges formed in the upper surface thereof.

FIGS. 8A and 8B illustrate front and side perspective views of another embodiment of the present invention showing a single cavity between the anterior and posterior portions of the implant with ridges in the upper surface thereof.

### DETAILED DESCRIPTION OF THE INVENTION

Reference will now be made in detail to embodiments of the invention, examples of which are illustrated in the accompanying drawings. Throughout the following detailed description, the same reference characters refer to the same or similar elements in all figures.

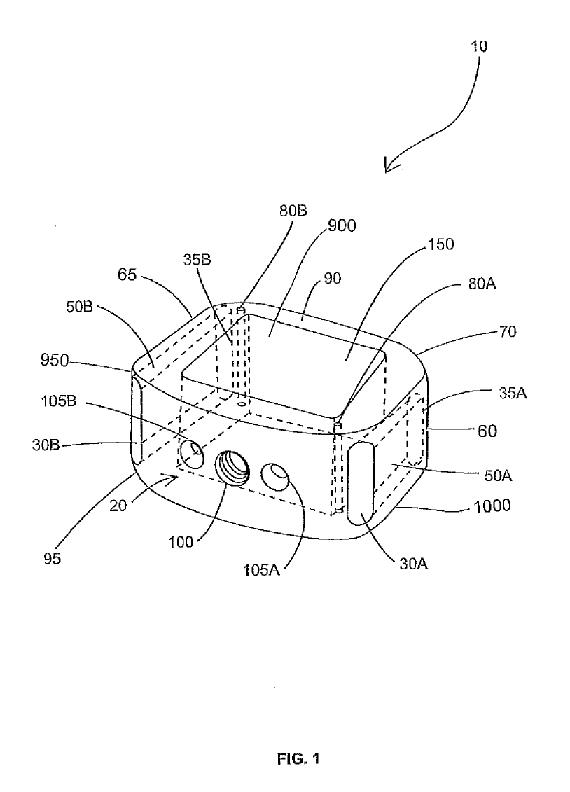

FIG. 1 depicts a front side perspective view of one embodiment of the present invention. The inter-vertebral implant 10 typically is disposed between two vertebrae, an inferior vertebral body below and in contact with the lower surface 95 of the inter-vertebral implant 10 and a superior vertebral body above and in contact with the upper surface 90 of the inter-vertebral implant 10.

The implant comprises a wall 1000 having a height chosen to correspond to the removed vertebral disc and the space that said that disc occupied. The anterior portion 20 of the implant 10 has orifices 30A and 30B, which are the anterior orifices for cavities 50A and 50B. The cavities 50A and 50B are contained within the first lateral portion 60 and the second lateral portion 65, respectively. The cavities 50A and 50B have second orifices 35A and 35B on the posterior portion 70 of the implant 10.

FIG. 1 depicts a detachment screw hole 100 and guide holes 105A and 1058. During surgery, the implant 10 is maneuvered into position by a detachable carrying wand. The screw hole 100 provides for attachment and detachment of the wand to and from the implant 10 such that it may be correctly positioned between the inferior and superior vertebral bodies.

FIG. 1 further depicts additional pinhole tubules 80A and 80B. These pinhole tubules 80A and 80B provide for insertion of detection materials used in various radiographic techniques, including x-ray radiography, nuclear magnetic resonance, and computer tomography.

FIG. 1 also depicts a bone-growth volume 150 in the center of the inter-vertebral implant 10. During surgery, the bone-growth volume 150 is typically packed with bone growth material to provide a means for fusion of the immediately adjacent superior and inferior vertebral bodies through the center of inter-vertebral implant. The bone growth volume 150 is enclosed by the inside surface 900 of the wall 1000. Importantly, the bone-growth volume 150 in no way contacts the cavities 50A and 50B, as the cavities and volume are separated and isolated by implant wall material. Thus, it is not possible for material in the bone growth volume and material in any cavity to intermix because they are separated by the inside surface 900 of the wall 1000 of the implant 10.

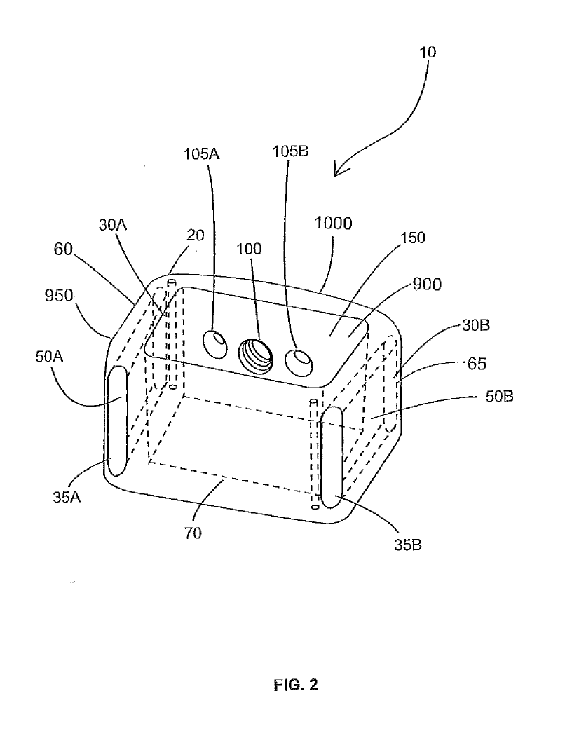

FIG. 2 depicts a backside perspective side view of one embodiment of the present invention. The posterior portion 70 is placed in contact with the spinal column region. Efflux of accumulating post surgical byproducts drains from the spinal column area through either or both posterior orifices 35A and 35B. The surgical byproduct material travels through the cavities 50A and 50B and eventually out the anterior portion cavity orifices 30A and 30B. Drainage of surgical byproduct material away from the spinal cord may be accomplished via suction and capillary action methods and internal pressure gradients. Additionally, treatments, including thrombotic agents, cooled saline, medicines and other materials may be delivered to the spinal column region through the anterior cavity orifice 30A and 30B, through the cavities 50A and 50B and through the posterior portion cavity orifices 35A and 35B. This provides a means to administer medicine directly to the space posterior to the inter-vertebral implant for the purpose of obtaining hemostasis or cooling the spinal cord.

In FIG. 1 and FIG. 2, the outside surface 950 of the wall 1000 comprises the anterior portion 20, the posterior portion 70, first lateral portion 60, and the second lateral portion 65. Such portions are integrally formed with the wall of the inter-vertebral device. Additionally, the anterior portion 20 and posterior portion 70 are attached to the first lateral portion 60 and the second lateral portion 65. Also, the first lateral portion 60 and the second lateral portion 65 each contain a cavities 50A and 50B.

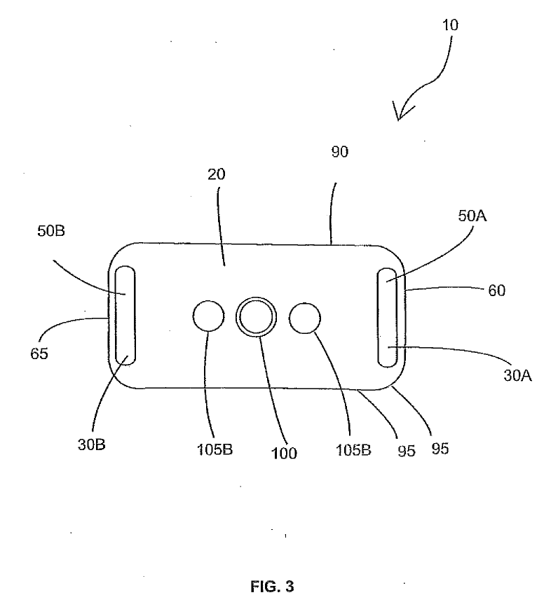

In FIG. 3, an front side planar view of the invention depicted in FIG. 1 and FIG. 2, the inter-vertebral implant 10 has two anterior portion cavity orifices 30A and 30B disposed on the anterior portion 20. The cavities 50A and 50B are disposed within the first lateral portion 60 and second lateral portion 65, respectively. FIG. 3 also depicts the guide holes 105A and 105B and the screw hole 100. The upper surface 90 and the lower surface 95 of the inter-vertebral implant 10 are also depicted with the anterior portion 20, the posterior portion 70, the first lateral portion 60, and the second lateral portion 65 disposed therebetween.

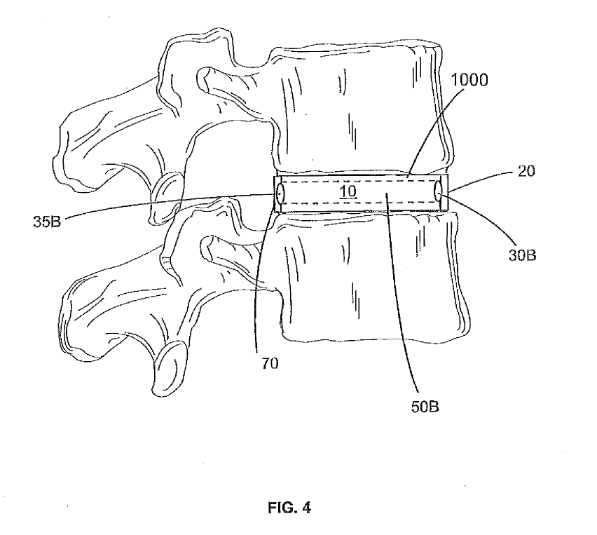

FIG. 4 is a side view showing the inter-vertebral device between two vertebrae, a superior vertebra and an inferior vertebra. FIG. 4 is depicted from the right-handed side of a patient and the posterior portion is near the spinal column, not depicted. The cavity 50B runs the length of the second lateral portion of the wall 1000 of the inter-vertebral implant. This allows material to flow between the cavity orifice 30B on the anterior portion 20 and the cavity orifice 35B on the posterior portion 70.

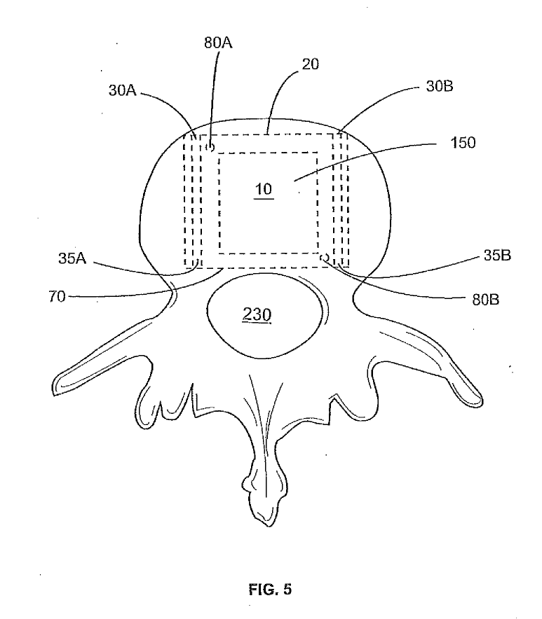

FIG. 5 illustrates a top planar view of one embodiment of the current invention disposed inside a cervical spine region. FIG. 5 depicts the invention disposed on an inferior vertebra. The posterior portion 70 of inter-vertebral implant 10 is in proximity to the spinal column 230. During and after surgery, a hemotoma may develop. Such hemotoma comprises surgical byproducts can drain via the cavities 50A and 50B contained within the inter-vertebral implant 10.

The current invention contemplates drainage cavities of various sizes and number to provide for the drainage of surgical byproducts and other materials as well as injection of medication or irrigation of the space posterior to the inter-vertebral implant. Cavity sizes range from centimeter-sized cavities to micron or even nanometer-sized cavities.

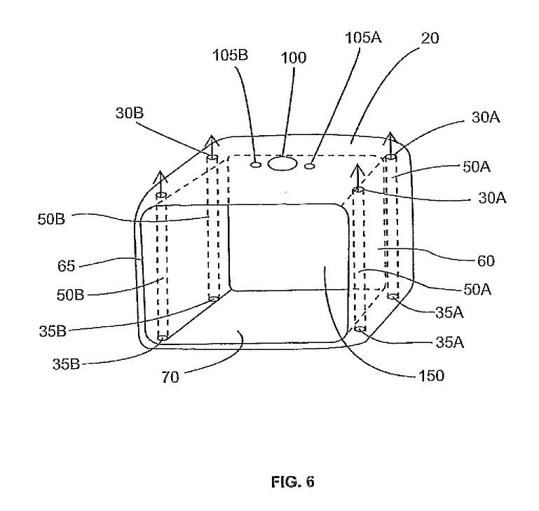

FIG. 6 illustrates an embodiment of the current invention, wherein there are multiple cavities 50A within the first lateral portion 60 and multiple cavities 50B within the second lateral portion 65.

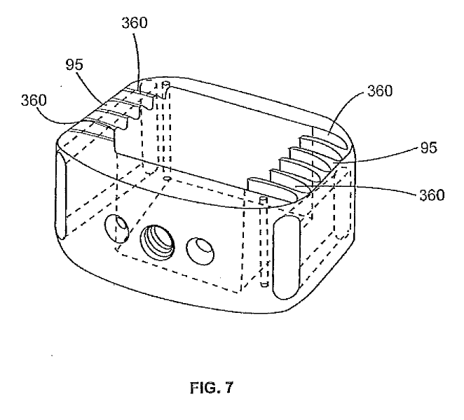

FIG. 7 depicts another embodiment of the current invention wherein ridges 360 are included on the lower surface 95. It is contemplated within the scope of this invention that both upper surface 90 and lower surface 95 of the inter-vertebral implant include such ridges 360.

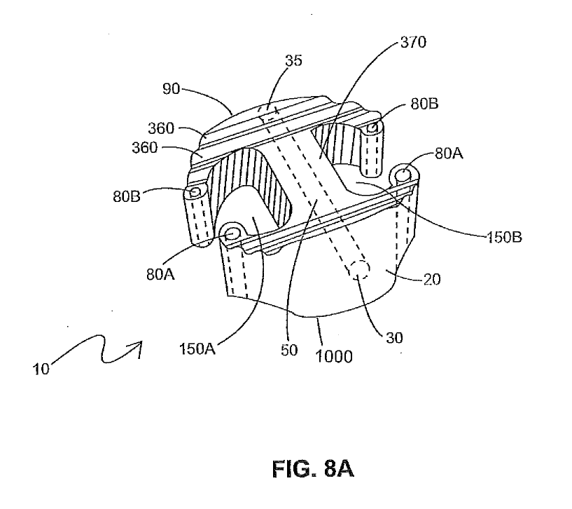

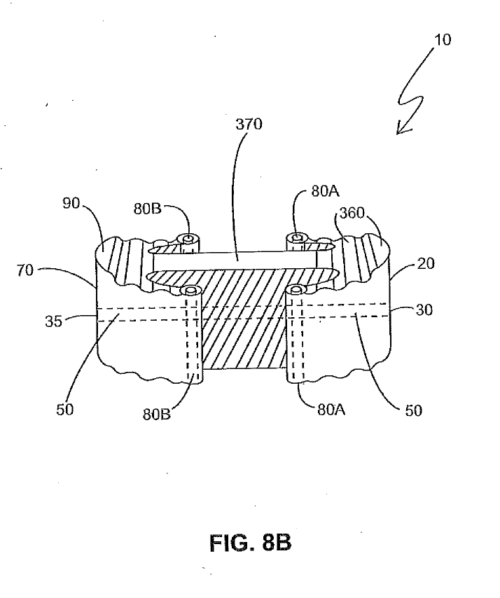

FIG. 8A and FIG. 8B depict another embodiment of the inter-vertebral implant of the present invention. FIG. 8A depicts a wall 1000 having a chosen height defining an upper surface 90 and a lower surface (not depicted). Said wall partially encloses two opposing bone growth volumes 150A and 150B. A portion of the wall, the medial portion 370 separates the opposing bone growth volumes 150A and 150B and has a cavity 50 therethrough having both an anterior orifice 30 in the anterior portion 20 and a posterior orifice 35 the posterior portion 70 of the wall 1000. Although not pictured, this invention includes other embodiments that include additional cavities through the wall separating the anterior portion 20 and posterior portion 70 of the wall 1000.

The current invention provides a means for the inter-vertebral implant 10 to have more peripheral contact with both the inferior and superior vertebral bodies on or about such vertebral body's apophyseal rings while still accommodating removal of surgical byproduct material from hemotoma in the vicinity of the spinal cord and delivery of material to the spinal cord region via the cavities of the implant. The additional peripheral contact between the implant and the vertebrae creates more stability, while the cavities still provide for the flow of surgical byproduct material away from the spinal cord and hemotoma and flow of materials to this region to aid healing. Thus, the current invention provides a means for a larger implant to have contact with the apophyseal ring and allow for more internal space for placing bone graft material in the bone growth volume. The apophyseal ring comprises strong bone and will resist subsidence of the implant into the bone when an axial load is applied through the implant.

Thus, the current invention will allow safe usage of the most optimally sized implant for the patient and yet still provide efflux drainage cavities for any post surgical blood products that may accumulate under pressure behind the implant. The cavities can be used to aspirate these products as they accumulate during surgery, and the cavities can be used to inject haemostatic agents behind the implant to prevent or stop any surgical byproducts that may lead to accumulation of post-surgical byproducts under pressure behind the implant. Alternatively the cavities maybe used to administer cold saline through one cavity and drain it through an additional cavity for the purpose of treating a spinal cord injury with hypothermic protocols.

One aspect of the invention, and in accordance with its objects and purposes, includes a method for reducing the accumulation of fluids in the vicinity of the spinal cord resulting from disc removal, includes the step of inserting an inter-vertebral implant comprising a wall having a chosen height defining an upper surface and a lower surface and partially enclosing two opposing bone growth volumes extending through the upper surface and the lower surface and separated by a portion of the wall, an outside surface of the wall forming an anterior portion, a posterior portion opposite thereto, the portion of the wall separating the opposing bone growth volumes having at least one cavity therethrough having an orifice in both the anterior portion and the posterior portion of the wall.

In another aspect of the invention, the invention includes a method for reducing the accumulation of fluids in the vicinity of the spinal cord resulting from disc removal hereof includes the step of inserting an inter-vertebral implant comprising a wall having a chosen height defining an upper surface and a lower surface and an inside surface enclosing a bone growth volume extending through the upper surface and lower surface, an outside surface of the wall forming an anterior portion, a posterior portion opposite thereto, a first lateral portion between the anterior portion and the posterior portion, and a second lateral portion between the anterior portion and the posterior portion opposite the first lateral portion, the first lateral portion having a cavity therethrough having an orifice in both the anterior portion and the posterior portion of the wall, and the second lateral portion having a cavity therethrough having an orifice in both the anterior portion and the posterior portion of the wall.

In another embodiment of the present invention, the method further comprises matching the size of the inter-vertebral implant to the size of the vertebrae such that there is nearly complete overlap between the implant and the surface of the vertebrae it is disposed between.

In another embodiment of the present invention the drainage cavities will function by either natural or mechanically applied pressure gradients from posterior to anterior or anterior to posterior direction as well as through the physical properties of capillary action.

It is believed that the apparatus of the present invention, and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The forms herein before described, being merely exemplary and explanatory embodiment thereof. It is the intention of the following claims to encompass and include such changes.

What is claimed is:

1. An inter-vertebral implant comprising: a wall having a chosen height defining an upper surface and a lower surface and an inside surface enclosing a bone growth volume extending through the upper surface and lower surface, an outside surface of the wall forming an anterior portion, a posterior portion opposite thereto, a first lateral portion between the anterior portion and the posterior portion, and a second lateral portion between the anterior portion and the posterior portion opposite the first lateral portion, the first lateral portion having a cavity therethrough having an orifice in both the anterior portion and the posterior portion of the wall, and the second lateral portion having a cavity therethrough having an orifice in both the anterior portion and the posterior portion of the wall.

2. The inter-vertebral implant of claim 1, wherein the anterior portion, posterior portion, the first lateral portion, and the second lateral portion are integrally formed.

3. The inter-vertebral body of claim 1, wherein the anterior portion, posterior portion are attached to the first lateral portion and the second lateral portion.

4. The inter-vertebral implant of claim 1 further comprising marking material sensitive to any of the following radiographic detection techniques: x-ray radiography, nuclear magnetic resonance, and computer tomography.

5. The inter-vertebral implant of claim 1, wherein the implant further comprises ridges along the upper surface.

6. The inter-vertebral implant of claim 1, wherein the implant further comprises ridges along the lower surface.

7. The inter-vertebral implant of claim 1, wherein the first lateral portion has at least one additional cavity.

8. The inter-vertebral implant of claim 1, wherein the second lateral portion has at least one additional cavity.

9. An inter-vertebral implant comprising a wall having a chosen height defining an upper surface and a lower surface, an internal surface partially enclosing two opposing bone growth volumes extending through the upper surface and the lower surface, a portion of the wall separating the bone growth volumes, and an outside surface of the wall forming an anterior portion, a posterior portion opposite thereto, the portion of the wall separating the opposing bone growth volumes having at least one cavity therethrough having an orifice in both the anterior portion and the posterior portion of the wall.

10. The inter-vertebral implant of claim 9, wherein the anterior portion, posterior portion, and portion of the wall separating the opposing bone growth volumes are integrally formed.

11. The inter-vertebral body of claim 9, wherein the anterior portion, posterior portion are attached to the portion of the wall separating the opposing bone growth volumes.

12. The inter-vertebral implant of claim 9 further comprising marking material sensitive to any of the following radiographic detection techniques: x-ray radiography, nuclear magnetic resonance, and computer tomography.

13. The inter-vertebral implant of claim 9, wherein the implant further comprises ridges along the upper surface.

14. The inter-vertebral implant of claim 9, wherein the implant further comprises ridges along the lower surface.

15. The inter-vertebral implant of claim 9, wherein the portion of the wall separating the opposing bone growth volumes has at least one additional cavity.

16. A method for reducing the accumulation of surgical byproduct material from the spinal cord and applying medicinal fluids in the vicinity of the spinal cord comprising the step of inserting an inter-vertebral implant between two vertebrae of the spinal column, the implant comprising a wall having a chosen height defining an upper surface and a lower surface, an inside surface of the wall at least partially enclosing one or more bone growth volumes extending through the upper surface and the lower surface, and an outside surface of the wall forming an anterior portion, a posterior portion opposite thereto, and a portion of the wall separating the anterior and posterior portions of the wall having at least one cavity therethrough having a orifices in both the anterior portion and the posterior portion of the wall.

17. The method of claim 16, wherein the anterior portion, posterior portion, and portion of the wall separating the anterior and posterior portions are integrally formed.

18. The method of claim 16, wherein the anterior portion and the posterior portion are attached by the portion of the wall separating the anterior and posterior portions.

19. The method of claim 16, wherein the implant further comprises marking material sensitive to any of the following radiographic detection techniques: x-ray radiography, nuclear magnetic resonance, and computer tomography.

20. The method of claim 16, wherein the portion of the wall separating the opposing bone growth volumes has at least one additional cavity.
